(19) United States
(12) Patent Application Publication
(10) Pub. No.: US 2017/0035509 A1
(43) Pub. Date: Feb. 9, 2017

(54) Puncturing Instrument, Puncturing Equipment, and Signal Processing Method of the Puncturing Equipment

(71) Applicants: Yin Chang, Taipei City (TW); METAL INDUSTRIES RESEARCH & DEVELOPMENT CENTRE, Kaohsiung City (TW)

(72) Inventors: Ming-Hui Chen, Kaohsiung City (TW); Yu-Min Ting, Kaohsiung City (TW); Ming-Hui Cheng, Kaohsiung City (TW); Yin Chang, Taipei City (TW)

(21) Appl. No.: 14/980,305

(22) Filed: Dec. 28, 2015

(30) Foreign Application Priority Data
Aug. 7, 2015 (TW) 104125815

(51) Int. Cl. A61B 18/22; A61B 17/34; A61B 18/20

(52) U.S. Cl. CPC A61B 18/22; A61B 18/20; A61B 18/201; A61B 17/34; A61B 2018/2244

(57) ABSTRACT

A puncturing instrument is provided to overcome the inconvenience in performing the puncturing operation due to the inability to see the internal structure of a patient. The puncturing instrument includes a first tube, a second tube and a light-guiding member is disclosed. The first tube includes two first openings. One of the two first openings forms a sharp portion. The second tube is received in the first tube and includes two second openings and a gas outlet. One of the two second openings is provided with a lens, and the gas outlet is adjacent to the lens and the sharp portion of the first opening. The light-guiding member is received in the second tube. Furthermore, a puncturing equipment using the puncturing instrument and a signal processing method of the puncturing equipment are also disclosed.

### CROSS REFERENCE TO RELATED APPLICATIONS

The application claims the benefit of Taiwan application serial No. 104125815, filed on Aug. 7, 2015, and the subject matter of which is incorporated herein by reference.

### BACKGROUND OF THE INVENTION

1. Field of the Invention

The present disclosure generally relates to a puncturing instrument and, more particularly, to a puncturing instrument that can be punctured into a body tissue.

2. Description of the Related Art

In the conventional surgery, a large incision is often formed on a body part. However, minimally invasive surgery (MIS) technology has been developed as the advance of the medical technology. In the minimally invasive surgery, a small incision instead of a large incision is formed on a body part. The minimally invasive surgery technology reduces not only the size of the wound but also the cost of the medical care. Thus, the period of hospitalization is significantly reduced.

As an example of the laparoscopic surgery, a conventional puncturing instrument (such as a veress needle) is inserted into the abdominal cavity of the patient from the belly skin, and air is pumped into the abdominal cavity to cause expansion of the abdominal cavity. Thus, the pneumoperitoneum procedure is complete. Then, the surgical instrucments are used to perform the surgery for the organs inside the abdominal cavity. One embodiment of such a conventional puncturing instrument can be seen in U.S. Pat. No. 5,098,388 entitled “Veress needle assembly” and U.S. Pat. No. 5,669,883 entitled “Veress needle and cannula assembly.”

The conventional puncturing instrument only has the inflation function. In this regard, the surgeon performs the puncturing operation in the abdominal cavity merely through the sense of operating the puncturing instrument and the experience without actually seeing the internal structure of the abdominal cavity. After the abdominal cavity is expanded by the pumped air, the surgical instrucments are inserted into the abdominal cavity to perform the surgery. However, when an unexpected situation (such as abdominal adhesion, which occurs at the possibility of smaller than 1%) is encountered if the patient hasn't had any abdominal surgery before, it is highly likely that the puncturing instrument injures the surrounding blood vessels and organs during the puncturing operation. As a result, bleeding may occur inside the body without the patient actually knowing it, leaving the patient in danger of losing their life. Thus, the puncturing operation is critical to the success of the surgery and determines whether there will be a complication or not.

In light of this, it is necessary to improve the conventional puncturing instrument.

### SUMMARY OF THE INVENTION

It is therefore the objective of this disclosure to provide a puncturing instrument that can detect the condition of a body tissue in an optical manner.

It is another objective of this disclosure to provide a puncturing equipment that can automatically recognize the condition of a body tissue.

It is a further objective of this disclosure to provide a signal processing method of the puncturing equipment which recognizes the condition of a body tissue by the absorbed reflected spectrum of the body tissue.

In an embodiment of the disclosure, a puncturing instrument including a first tube, a second tube and a light-guiding member is disclosed. The first tube includes two first openings. One of the two first openings forms a sharp portion. The second tube is received in the first tube and includes two second openings and a gas outlet. One of the two second openings is provided with a lens, and the gas outlet is adjacent to the lens and the sharp portion of the first opening. The light-guiding member is received in the second tube.

In the disclosure, a puncturing equipment including the puncturing instrument and a computer system is disclosed. The puncturing instrument includes a first tube, a second tube and a light-guiding member. The first tube includes two first openings. One of the two first openings forms a sharp portion. The second tube is received in the first tube and includes two second openings and a gas outlet. One of the two second openings is provided with a lens, and the gas outlet is adjacent to the lens and the sharp portion of the first opening. The light-guiding member is received in the second tube. The computer system is coupled with the light-guiding member of the puncturing instrument and controls a light source to transmit light to the light-guiding member. The light-guiding member receives a reflected spectrum, compares the reflected spectrum with a reference spectrum, and determines a difference between the reflected spectrum and the reference spectrum. The computer system issues a warning message if the difference is larger than a threshold, or receives another reflected spectrum if the difference is not larger than the threshold.

In a form shown, the light-guiding member may include a plurality of parallel fibers that is bonded together as a beam of fiber.

In the form shown, the plurality of parallel fibers includes a first fiber and a plurality of second fibers. The first fiber is adapted to transmit light to the lens. The plurality of second fibers includes outer surfaces coupled with an outer surface of the first fiber, so as to receive the light from the lens.

In the form shown, a first engaging portion may be arranged on an inner surface of the second tube, and the light-guiding member may include a second engaging portion engaged with the first engaging portion. The first engaging portion may be adjacent to the lens.

In the form shown, a first guiding portion may be arranged on an inner surface of the second tube and may extend along a longitudinal axis of the second tube. A second guiding portion is arranged on an outer surface of the light-guiding member. The second guiding portion may be slidably engaged with the first guiding portion.

In the disclosure, a signal processing method of the puncturing equipment is disclosed. The signal processing method may be executed by a computer system and includes receiving a reflected spectrum, comparing the reflected spectrum with a reference spectrum to determine a difference therebetween, and issuing a warning message if the difference is larger than a threshold or receiving another reflected spectrum if the difference is not larger than the threshold.

In another form shown, the threshold is a waist threshold. The reference spectrum includes a first waveform with a first maximum value, and a width of the first waveform at half of the first maximum value is defined as a reference waistline. The reflected spectrum includes a second waveform with a second maximum value, and a width of the second waveform at half of the second maximum value is defined as a reflected waistline. The computer system issues the warning message if the difference between the reference waistline and the reflected waistline is larger than the waist threshold.

In the other form shown, the threshold is a peak threshold. The reference spectrum includes a first waveform having a first peak, a second peak, a third peak, a fourth peak and a fifth peak from left to right. The reflected spectrum includes a second waveform having a first peak, a second peak, a third peak, a fourth peak and a fifth peak from left to right. The computer system issues the warning message if a difference between the first peaks of the reference spectrum and the reflected spectrum, between the second peaks of the reference spectrum and the reflected spectrum, or between the third peaks of the reference spectrum and the reflected spectrum, is larger than the peak threshold.

In the other form shown, the computer system is connected to the light-guiding member of the puncturing instrument through a light cable. The light cable is provided with a light emitting element and at least one image retrieving element. The light emitting element emits the light towards the light-guiding member, and the at least one image retrieving element retrieves the reflected spectrum from the light-guiding member.

In the other form shown, the puncturing instrument further includes an air valve coupled to one end of the puncturing equipment opposite to the gas outlet.

In the other form shown, the puncturing instrument further includes air valve coupled to one end of the puncturing equipment opposite to the gas outlet, so as to regulate an amount of air.

In the other form shown, the puncturing instrument further includes a sheath coupled with an outer surface of the puncturing instrument.

The reference spectrum may be pre-stored in the computer system.

In the above puncturing instrument, the puncturing equipment, and the signal processing method of the puncturing equipment, the optical response of the body tissue can be observed in real time. The observed result may be compared with the reference spectrum. In this regard, a warning message may be issued if an abnormality has been found in order not to cause the internal bleeding of the patient resulting from the injury of the surrounding blood vessels and organs. Advantageously, the probability of a successful puncturing operation can be increased, and the probability of the failure and the incidence of a complication can be reduced. Thus, the puncturing instrument, the puncturing equipment, and the signal processing method of the puncturing equipment according to the embodiment of the disclosure are suitable for use in medical care.

### BRIEF DESCRIPTION OF THE DRAWINGS

The present disclosure will become more fully understood from the detailed description given hereinafter and the accompanying drawings which are given by way of illustration only, and thus are not limitative of the present disclosure, and wherein:

FIG. 1 is a cross sectional view of a puncturing instrument according to a first embodiment of the disclosure.

FIG. 2 is an enlarged cross sectional view of a light-guiding member of the puncturing instrument shown in FIG. 1.

FIG. 3 shows the use of a puncturing equipment according to a second embodiment of the disclosure.

FIG. 4a shows a reference spectrum according to the first and second embodiments of the disclosure.

FIG. 4b shows a reflected spectrum according to the first and second embodiments of the disclosure.

In the various figures of the drawings, the same numerals designate the same or similar parts. Furthermore, when the terms “first”, “second”, “third”, “fourth”, “inner”, “outer”, “top”, “bottom”, “front”, “rear” and similar terms are used hereinafter, it should be understood that these terms have reference only to the structure shown in the drawings as it would appear to a person viewing the drawings, and are utilized only to facilitate describing the disclosure.

### DETAILED DESCRIPTION OF THE INVENTION

The term “coupling” refers to the intercommunication between two devices via the optical technology, such as the connection between an optical fiber and an optical cable, as it can be readily appreciated by the persons skilled in the art. However, this is not taken as a limited sense.

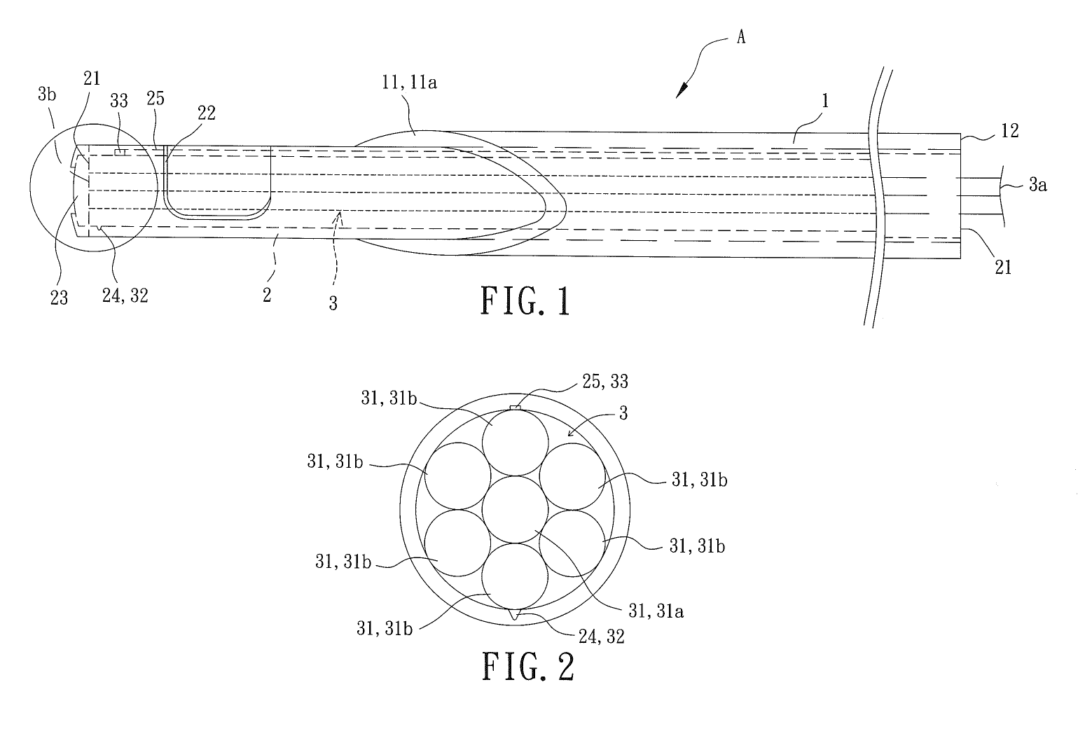

FIG. 1 is a cross sectional view of a puncturing instrument according to an embodiment of the disclosure. The puncturing instrument “A” according to the embodiment may include a first tube 1, a second tube 2 and a light-guiding member 3. The second tube 2 is received in the first tube 1. The light-guiding member 3 is received in the second tube 2.

Referring to FIG. 1, the first tube 1 may be made of a rigid material (such as metal or plastic) and may include two first openings 11 and 12. The first opening 11 may form a sharp portion 11a that can penetrate into a body tissue. In the embodiment, the first tube 1 may be made of a metal material. The sharp portion 11a may, form a sharp shape by a blade cutting the first opening 11. The shape of the sharp portion 11a is not limited to what is shown in the drawing. An object may be placed into the first tube 1 via the first opening 12, or gas may flow into the first tube 1 via the first opening 12. However, the access to the first opening 12 is not limited to the object and the gas.

Referring to FIG. 1 again, the second tube 2 may be made of a rigid material (such as metal or plastic) and may include two second openings 21 and a gas outlet 22. One of the second openings 21 is provided with a lens 23. The gas outlet 22 is adjacent to the lens 23 and the sharp portion 11a of the first opening 11. In the embodiment, the gas outlet 22 can slide out of the sharp portion 11a of the first opening 11 to send the gas into a space (such as an internal space of the human body) via the second tube 2 moving relative to the first tube 1. The lens 23 may be an optical lens having excellent light permeability. A first engaging portion 24 may be arranged on an inner surface of the second tube 2 (such as a hole) for engaging the light-guiding member 3. The first engaging portion 24 may be adjacent to the lens 23 (such as a convex lens). However, the location of the first engaging portion 24 is not limited thereto. A first guiding portion 25 may be arranged on the inner surface of the second tube 2 (such as a guiding groove). The first guiding portion 25 extends along a longitudinal axis of the second tube 2 in order to guide the light-guiding member 3 into the second tube 2. However, this is not taken as a limited sense.

Referring to FIG. 1 again, the light-guiding member 3 is made of a material capable of transmitting light, such as an optical fiber. In this regard, the light of a light source may be transmitted from one end 3a to another end 3b. Then, the reflected light may be received by the end 3b and transmitted to the end 3a. In the embodiment, the light-guiding member 3 includes a plurality of parallel fibers 31 that is bonded together (such as by adhesive) as a beam of fiber, as shown in FIG. 2. The beam of fiber may include a first fiber 31a and a plurality of second fibers 31b. The first fiber 31a is used to transmit the light of the light source to the lens 23. The outer surfaces of the plurality of second fibers 31b may couple with the outer surface of the first fiber 31a, so that the plurality of second fibers 31b is able to receive the light of the light source. The plurality of second fibers 31b may evenly surround the first fiber 31a to enhance the light receiving efficiency. The quantity of the plurality of second fibers 31b may be 6, and the material and size of the plurality of second fibers 31b may be different from the first fiber 31a. However, this is not taken as a limited sense. Furthermore, the light-guiding member 3 may include a second engaging portion 32 (such as a protrusion), which may be engaged with the first engaging portion 24 of the second tube 2. The first engaging portion 24 and the second engaging portion 32 may be of any structures that can be engaged with each other. Moreover, the light-guiding member 3 may include a second guiding portion 33 (such as a guiding projection) that can be slidably engaged with the first guiding portion 25 of the second tube 2. The first guiding portion 25 and the second guiding portion 33 can be of any structures that can be slidably engaged with each other.

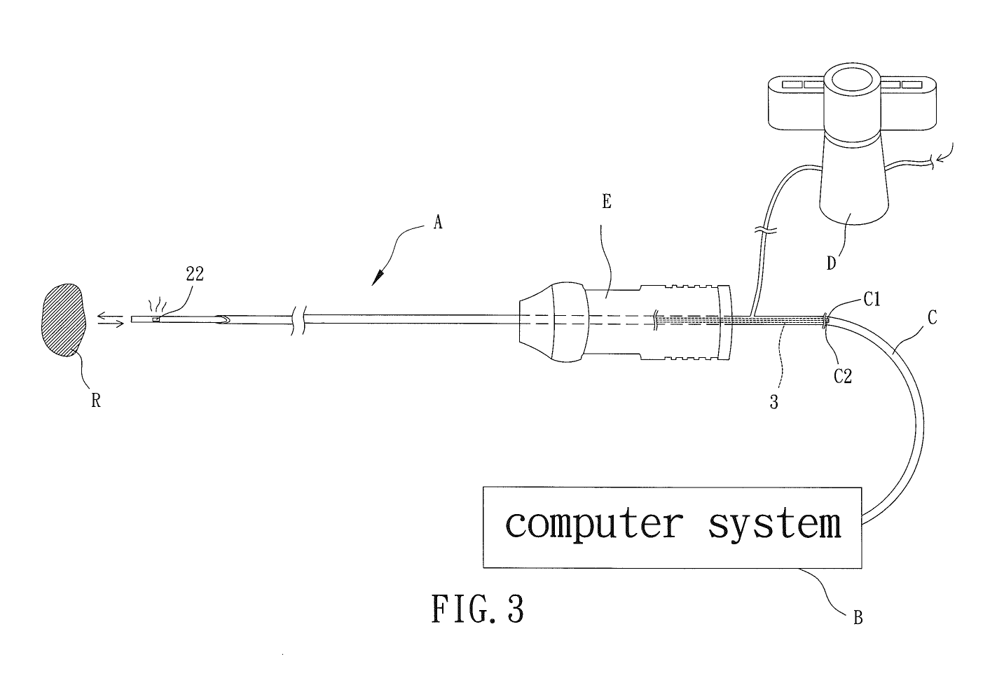

FIG. 3 shows the use of a puncturing equipment according to the disclosure. The puncturing equipment may be formed by the puncturing instrument according to the embodiment of the disclosure. The puncturing equipment includes the puncturing instrument “A” and a computer system “B.” The puncturing instrument “A” has already been described before. The computer system “B” may couple with the light-guiding member 3 of the puncturing instrument “A.” The computer system “B” may control the light transmission of the light source along the first fiber 31a. In this regard, a reflected spectrum may be received by the plurality of second fibers 31b of the light-guiding member 3 and is compared with a reference spectrum (which may be pre-stored in the computer system “B”) to determine a difference therebetween. If the difference is larger than a threshold, a warning message is issued. If the difference is not larger than the threshold, another reflected spectrum is received. In the disclosure, the computer system “B” may be a device capable of processing light signals or electricity signals, such as a photoelectric and microcomputer system. Alternatively, the computer system “B” may be formed by a light emitting element, an image retrieving element, a human machine interface, a signal processor and a database. The computer system “B” may store a software program that is used to perform the signal processing. Before the reflected spectrum is compared with the reference spectrum, the waveform of the reflected spectrum may be normalized to remove the abnormal signals or noises. However, this is not taken as a limited sense. The computer system “B” may also store the reference spectrum, which may be updated by the user's input. Furthermore, the computer system “B” may be connected to the light-guiding member 3 of the puncturing instrument “A” through a light cable “C.” One end of the light cable “C” (connected to the light-guiding member 3) may be provided with a light emitting element C1 (such as a white light-emitting diode) and at least one image retrieving element C2 (such as a charge-coupled device (CCD) or a micro lens). The light emitting element C1 can emit the light towards the light-guiding member 3. The at least one image retrieving element C2 can retrieve the reflected spectrum from the light-guiding member 3. The light cable “C” and the light-guiding member 3 may be engaged with each other by an engaging assembly (formed by a male tenon and a female tenon, which are not shown in the drawings). The puncturing equipment may further include an air valve “D” (such as a conventional fluid regulating valve) that is coupled to one end of the puncturing equipment opposite to the gas outlet 22. The air valve “D” is used to regulate the amount of air, as it can be readily appreciated by the skilled persons. Furthermore, the puncturing equipment may also include a sheath “E” coupled with the outer surface of the puncturing instrument “A.” The shape of the sheath “E” can be changed as desired according to the user's requirement.

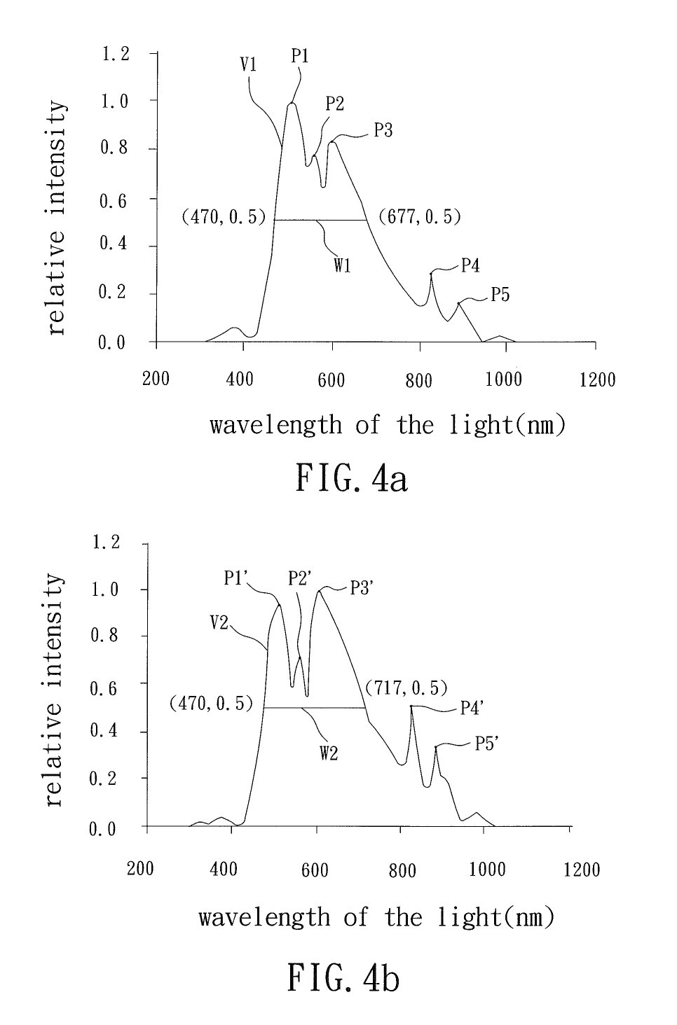

FIGS. 4a and 4b show the reference spectrum and the reflected spectrum of the disclosure. It can be observed from FIG. 4a that the waveform V1 of the reference spectrum includes a first peak P1, a second peak P2, a third peak P3, a fourth peak P4 and a fifth peak P5 from left to right. The width of the waveform V1 at half of the maximum value (about the relative intensity of 0.5) can be defined as a reference waistline W1 (about 470 nm to 677 nm). It can be observed from FIG. 4b that the waveform V2 of the reflected spectrum includes a first peak P1′, a second peak P2′, a third peak P3′, a fourth peak P4′ and a fifth peak P5′ from left to right. The width of the waveform V2 at half of the maximum value (about the relative intensity of 0.5) can be defined as a reflected waistline W2 (about 470 nm to 717 nm). The value of the reflected waistline W2 is not fixed, but is larger than the normal value.

Referring to FIGS. 4a and 4b again, if the difference between the reference waistline W1 and the reflected waistline W2 (such as (717−470)−(677−470)=40) is larger than the threshold (such as a waist threshold of 15), the computer system “B” may issue an warning message in the form of light, sound or text). Alternatively, if the difference between the first peaks P1 and P1′ is larger than the threshold, the difference between the second peaks P2 and P2′ is larger than the threshold, or the difference between the third peaks P3 and P3′ is larger than the threshold (the peak thresholds of the first, second and third peaks may be set as 0), the computer system “B” may issue an warning message. However, this is not taken as a limited sense.

Referring to FIGS. 1, 2 and 3 again, when the puncturing equipment performs the puncturing operation (such as the laparoscopic operation), the sharp portion 11a of the first tube 1 may be punctured into the abdomen of the human body (not shown). At this time, the computer system “B” can control the light emitting element C1 to transmit the light to a body tissue “R” via the light-guiding member 3. The reflected spectrum may be sent to the computer system “B” for analysis via the light-guiding member 3. If an unexpected situation (such as abdominal adhesion) occurs, the computer system “B” may issue a warning message immediately. Thus, the surgeon can stop the puncturing operation, thus preventing the injury of the surrounding blood vessels and organs as well as the internal bleeding of the patient. As such, the probability of a successful puncturing operation can be increased, and the probability of the failure and the incidence of a complication can be reduced.

Besides, a signal processing method of the puncturing equipment is disclosed according to the disclosure and is executed by the computer system “B.” The method includes receiving the reflected spectrum, determining a difference between the reflected spectrum and the reference spectrum, and issuing a warning message if the difference is larger than a threshold or receiving another reflected spectrum if the difference is not larger than the threshold. The details have been described before so they are not discussed herein again.

The puncturing instrument, the puncturing equipment, and the signal processing method of the puncturing equipment have the following characteristics. The second tube of the puncturing instrument is received in the first tube, and the light-guiding member is received in the second tube. The computer system is coupled with the light-guiding member of the puncturing instrument to control the light transmission from the light source to the light-guiding member. The computer system further receives the reflected spectrum via the light-guiding member and determines a difference between the reflected spectrum and the reference spectrum. If the difference is larger than a threshold, a warning message is issued. Moreover, the puncturing instrument according to the embodiment of the disclosure may be integrated in an existing puncturing equipment. Based on this, when the puncturing equipment is used to perform the puncturing operation, the computer system may issue a warning message if an unexpected situation (such as abdominal adhesion) occurs. Thus, the surgeon can stop the puncturing operation immediately in order not to cause the internal bleeding of the patient resulting from the injury of the surrounding blood vessels and organs. Advantageously, the probability of a successful puncturing operation can be increased, and the probability of the failure and the incidence of a complication can be reduced.

Based on the above, in the puncturing instrument, the puncturing equipment, and the signal processing method of the puncturing equipment, the optical response of the body tissue can be observed in real time. The observed result may be compared with the reference spectrum. In this regard, a warning message may be issued if an abnormality has been found in order not to cause the internal bleeding of the patient resulting from the injury of the surrounding blood vessels and organs. Advantageously, the probability of a successful puncturing operation can be increased, and the probability of the failure and the incidence of a complication can be reduced. Thus, the puncturing instrument, the puncturing equipment, and the signal processing method of the puncturing equipment according to the disclosure are suitable for use in medical care.

Although the disclosure has been described in detail with reference to its presently preferable embodiments, it will be understood by one of ordinary skill in the art that various modifications can be made without departing from the spirit and the scope of the disclosure, as set forth in the appended claims.

What is claimed is:

1. A puncturing instrument comprising:
a first tube comprising two first openings, wherein one of the two first openings forms a sharp portion;
a second tube received in the first tube and comprising two second openings and a gas outlet, wherein one of the two second openings is provided with a lens, and wherein the gas outlet is adjacent to the lens and the sharp portion of the first opening; and
a light-guiding member received in the second tube.

2. The puncturing instrument as claimed in claim 1, wherein the light-guiding member comprises a plurality of parallel fibers that is bonded together as a beam of fiber.

3. The puncturing instrument as claimed in claim 2, wherein the plurality of parallel fibers comprises a first fiber and a plurality of second fibers, wherein the first fiber is adapted to transmit light to the lens, and wherein the plurality of second fibers comprises outer surfaces coupled with an outer surface of the first fiber, so as to receive the light from the lens.

4. The puncturing instrument as claimed in claim 1, wherein a first engaging portion is arranged on an inner surface of the second tube, wherein the light-guiding member comprises a second engaging portion engaged with the first engaging portion, and wherein the first engaging portion is adjacent to the lens.

5. The puncturing instrument as claimed in claim 1, wherein a first guiding portion is arranged on an inner surface of the second tube and extends along a longitudinal axis of the second tube, wherein a second guiding portion is arranged on an outer surface of the light-guiding member, and wherein the second guiding portion is slidably engaged with the first guiding portion.

6. A puncturing equipment comprising:
a puncturing instrument comprising a first tube, a second tube and a light-guiding member, wherein the first tube comprises two first openings, wherein one of the two first openings forms a sharp portion, wherein the second tube is received in the first tube and comprises two second openings and a gas outlet, wherein one of the two second openings is provided with a lens, wherein the gas outlet is adjacent to the lens and the sharp portion of the first opening, and wherein the light-guiding member received in the second tube; and
a computer system coupled with the light-guiding member of the puncturing instrument and controlling a light source to transmit light to the light-guiding member, wherein the light-guiding member receives a reflected spectrum, wherein the computer system compares the reflected spectrum with a reference spectrum and determines a difference between the reflected spectrum and the reference spectrum, and wherein the computer system issues a warning message if the difference is larger than a threshold, or receives another reflected spectrum if the difference is not larger than the threshold.

7. The puncturing equipment as claimed in claim 6, wherein the threshold is a waist threshold, wherein the reference spectrum has a first waveform with a first maximum value, wherein a width of the first waveform at half of the first maximum value is defined as a reference waistline, wherein the reflected spectrum has a second waveform with a second maximum value, wherein a width of the second waveform at half of the second maximum value is defined as a reflected waistline, and wherein the computer system issues the warning message if the difference between the reference waistline and the reflected waistline is larger than the waist threshold.

8. The puncturing equipment as claimed in claim 6, wherein the threshold is a peak threshold, wherein the reference spectrum comprises a first waveform having a first peak, a second peak, a third peak, a fourth peak and a fifth peak from left to right, wherein the reflected spectrum comprises a second waveform having a first peak, a second peak, a third peak, a fourth peak and a fifth peak from left to right, and wherein the computer system issues the warning message if a difference between the first peaks of the reference spectrum and the reflected spectrum is larger than the peak threshold.

9. The puncturing equipment as claimed in claim 6, wherein the threshold is a peak threshold, wherein the reference spectrum comprises a first waveform having a first peak, a second peak, a third peak, a fourth peak and a fifth peak from left to right, wherein the reflected spectrum comprises a second waveform having a first peak, a second peak, a third peak, a fourth peak and a fifth peak from left to right, and wherein the computer system issues the warning message if a difference between the second peaks of the reference spectrum and the reflected spectrum is larger than the peak threshold.

10. The puncturing equipment as claimed in claim 6, wherein the threshold is a peak threshold, wherein the reference spectrum comprises a first waveform having a first peak, a second peak, a third peak, a fourth peak and a fifth peak from left to right, wherein the reflected spectrum comprises a second waveform having a first peak, a second peak, a third peak, a fourth peak and a fifth peak from left to right, and wherein the computer system issues the warning message if a difference between the third peaks of the reference spectrum and the reflected spectrum is larger than the peak threshold.

11. The puncturing equipment as claimed in claim 6, wherein the computer system is connected to the light-guiding member of the puncturing instrument through a light cable, wherein the light cable is provided with a light emitting element and at least one image retrieving element, wherein the light emitting element emits the light towards the light-guiding member, and wherein the at least one image retrieving element retrieves the reflected spectrum from the light-guiding member.

12. The puncturing equipment as claimed in claim 6, further including an air valve coupled to one end of the puncturing equipment opposite to the gas outlet.

13. The puncturing equipment as claimed in claim 6, further including a sheath coupled with an outer surface of the puncturing instrument.

14. The puncturing equipment as claimed in claim 6, wherein the light-guiding member comprises a plurality of parallel fibers that is bonded together as a beam of fiber.

15. The puncturing equipment as claimed in claim 14, wherein the plurality of parallel fibers comprises a first fiber and a plurality of second fibers, wherein the first fiber is adapted to transmit the light to the lens, and wherein the plurality of second fibers comprises outer surfaces coupled with an outer surface of the first fiber, so as to receive the light from the lens.

16. The puncturing equipment as claimed in claim 6, wherein a first engaging portion is arranged on an inner surface of the second tube, wherein the light-guiding member comprises a second engaging portion engaged with the first engaging portion, and wherein the first engaging portion is adjacent to the lens.

17. The puncturing equipment as claimed in claim 6, wherein a first guiding portion is arranged on an inner surface of the second tube and extends along a longitudinal axis of the second tube, wherein a second guiding portion is arranged on an outer surface of the light-guiding member, and wherein the second guiding portion is slidably engaged with the first guiding portion.

18. A signal processing method of a puncturing equipment, as executed by a computer system, comprising:
receiving a reflected spectrum;
comparing the reflected spectrum with a reference spectrum to determine a difference therebetween; and
issuing a warning message if the difference is larger than a threshold or receiving another reflected spectrum if the difference is not larger than the threshold.

19. The signal processing method of the puncturing equipment as claimed in claim 18, wherein the threshold is a waist threshold, wherein the reference spectrum comprises a first waveform with a first maximum value, wherein a width of the first waveform at half of the first maximum value is defined as a reference waistline, wherein the reflected spectrum comprises a second waveform with a second maximum value, wherein a width of the second waveform at half of the second maximum value is defined as a reflected waistline, and wherein the computer system issues the warning message if the difference between the reference waistline and the reflected waistline is larger than the waist threshold.

20. The signal processing method of the puncturing equipment as claimed in claim 18, wherein the threshold is a peak threshold, wherein the reference spectrum comprises a first waveform having a first peak, a second peak, a third peak, a fourth peak and a fifth peak from left to right, wherein the reflected spectrum comprises a second waveform having a first peak, a second peak, a third peak, a fourth peak and a fifth peak from left to right, and wherein the computer system issues the warning message if a difference between the first peaks of the reference spectrum and the reflected spectrum is larger than the peak threshold.

21. The signal processing method of the puncturing equipment as claimed in claim 18, wherein the threshold is a peak threshold, wherein the reference spectrum comprises a first waveform having a first peak, a second peak, a third peak, a fourth peak and a fifth peak from left to right, wherein the reflected spectrum comprises a second waveform having a first peak, a second peak, a third peak, a fourth peak and a fifth peak from left to right, and wherein the computer system issues the warning message if a difference between the second peaks of the reference spectrum and the reflected spectrum is larger than the peak threshold.

22. The signal processing method of the puncturing equipment as claimed in claim 18, wherein the threshold is a peak threshold, wherein the reference spectrum comprises a first waveform having a first peak, a second peak, a third peak, a fourth peak and a fifth peak from left to right, wherein the reflected spectrum comprises a second waveform having a first peak, a second peak, a third peak, a fourth peak and a fifth peak from left to right, and wherein the computer system issues the warning message if a difference between the third peaks of the reference spectrum and the reflected spectrum is larger than the peak threshold.

23. The signal processing method of the puncturing equipment as claimed in claim 18, wherein the reference spectrum is pre-stored in the computer system.
